(19) United States
(12) Patent Application Publication
(10) Pub. No.: US 2007/0276392 A1
(43) Pub. Date: Nov. 29, 2007

(54) Soft Tissue to Bone Fixation

(75) Inventors: Mordechay Beyar, Caesarea (IL); Yohanan Chorev, Raanana (IL); Ronen Shavit, Tel-Aviv (IL); Hila Wachsler-Avrahami, Tel-Aviv (IL)

Correspondence Address: William H dippert;Wolf Block Schorr & Solis-Cohen, 250 Park Avenue, New York, NY, 10177, US

(73) Assignee: DISC-O-TECH MEDICAL TECHNOLOGIES LTD, HERZELIA (IL)

(21) Appl. No.: 10/575,003

(22) PCT Filed:
(86) PCT No.: PCT/IL04/00923

(51) Int. Cl. A61B 17/58

(52) U.S. Cl. 606/80; 606/151; 606/79

(57) ABSTRACT

A device for forming expansion regions in a bone, comprising, an elongate body having and axis and adapted to fit through a tunnel in a bone; and a head, provided on said body and having outer dimensions not substantially extending trans-axially to said body, wherein said head is rotatable and wherein said head is adapted to widen said tunnel in said bone by at least 10% in conjunction with said rotating.

### RELATED APPLICATIONS

The present application claims the benefit under 119(e) of US Provisional Application No. 60/508,900, filed Oct. 7, 2003, the disclosure of which is incorporated herein by reference.

### FIELD OF THE INVENTION

The present invention relates to fixation of soft tissue to bone and to forming hollows in bone, for example, for the reconstruction/replacement of damaged ligaments and/or tendons.

### BACKGROUND OF THE INVENTION

In the human body, bones at a joint are typically connected with a ligament or tendon. As a result of strenuous activity or an accident, a ligament or tendon may be severed or detached from the bones. Ligaments are a band of fibrous tissues connecting bones or cartilage and serve to support and strengthen joints. Tendons are fibrous tissues which connect muscles to bones or cartilage (from Dorland's medical dictionary). Replacement of severed or detached tendons/ligaments is typically performed by fixation of the soft tissue of the tendon/ligament to bone using mechanical anchoring devices such as a screw, staple, pin, spring or dowel. In the present application where the word ligament is used, it is intended to include also tendons, unless otherwise noted.

An example of damage requiring repair is a rupture of the anterior cruciate ligament (ACL) of the knee. ACL reconstruction is generally performed by removing the damaged ligament and replacing it with another ligament or tendon. The replacement ligament or tendon may be of an autogenic origin, for example a hamstring tendon; allogenic origin, for example a cadaver Achilles tendon; or a synthetic artificial replacement, for example Dacron.

Typically, a graft tendon is harvested with pieces of bone at its extremities, for example a “bone tendon bone” (BTB) graft from the patellar tendon. A through hole is drilled from the upper part of the tibia through the tibia and into to the lower part of the femur, close to the original attachment points of the ACL. The graft is inserted through the hole and secured at either end, to the tibia and femur respectively. The bone sections may assist in fixation.

The method of securing the graft to the bone can significantly impact the long-term effectiveness of the successful reconstruction of the ACL. In some cases, the ends of the graft are fixated to the bone by a further invasive procedure, such as inserting screws or other fastening devices laterally through the walls of the femur (e.g., to the hole) and/or the tibia. The fastening devices may be metal (e.g. stainless steel or titanium) or a bio-absorbable polymer (e.g. polylactide). The use of fastening devices may lead to complications such as damage to the graft and/or mispositioning. Additionally, insertion of fastening devices requires further incisions in the patient and can cause additional trauma to the bone. The use of metal fasteners may hinder the use of visualization devices such as MRI or CT in evaluation of the patient. The use of bio-absorbable fasteners may encounter other difficulties such as breakage during insertion or after a relative short period of time before complete connection between the ligament and the bone. Additionally, the use of bio-absorbable fasteners may require extra preparation of the insertion path, which lengthens the insertion procedure. Another problem with insertion of an external fastener is the complication of aiming an external fastener through the bone to accurately interface the graft inside the bone. This may require a special device. Not all of these problems are critical.

U.S. Pat. No. 6,325,804 to Wenstrom et al. the disclosure of which is incorporated herein by reference, describes an ACL reconstruction wherein an adhesive is used to secure a bone plug attached to a ligament or tendon in a tunnel in the femora.

### SUMMARY OF THE INVENTION

An aspect of some embodiments of the invention, relates to a method of connecting a replacement ligament or tendon to a bone. In an exemplary embodiment of the invention, a single hole is drilled into the bone, such that the diameter of the hole is greater distal from the entry into the bone than is the opening. An end of the replacement ligament is inserted into the hole and anchored in place by filling the hole or a portion thereof with a plug that prevents the ligament from exiting the hole. Optionally, the plug is formed of a flowable material that sets to a hardened condition. Optionally, the material is an adhesive that adheres to the ligament. Alternatively, a plug is used, for example a plug formed of a resilient deformable material such as rubber.

In an exemplary embodiment of the invention, the ligament is prevented from retracting out of the hole by mechanical interference, i.e., the ligament and the plug together have a diameter greater than the proximal diameter of the hole. Optionally or additionally the adhesive serves to attach the ligament and/or the plug to the bone.

In an exemplary embodiment of the invention, the other end of the ligament or tendon is fastened to a second bone in a similar manner or by methods known in the art, for example using a pin or screw. In some embodiments of the invention, the replaced ligament is an anterior cruciate ligament (ACL). Optionally, a single hole is created in the femur and the other end is attached to the tibia. In some embodiments of the invention, the replaced ligament is a posterior cruciate ligament (PCL).

An aspect of some embodiments of the invention, relates to hole forming apparatus adapted for drilling a hole in a bone having a greater distal diameter than its adjacent proximal portion diameter.

In some embodiments of the invention, the hole forming apparatus has two operational modes, a hole drilling mode, in which a hole of a first diameter is formed and a reaming mode in which a void of a greater diameter is formed by reaming out the hole. In some embodiments of the invention, the mode is selected automatically, for example, by the direction of rotation of the hole forming apparatus selecting the mode. Alternatively, the mode is user selectable, for example using a switch or a selection pin. In some embodiments of the invention, the mode is selected by axial motion of the apparatus.

In an exemplary embodiment of the invention, the hole forming apparatus comprises an axial hollow sleeve and a head having a shaft that fits in the sleeve hollow. Reaming is provided by the shaft rotating in the hollow in a way that the head does not rotate around the axis of the sleeve, thus exhibiting eccentric reaming action. Optionally, switching between modes is provided by relative trans-axial motion of the shaft and the sleeve. Optionally, the head is mounted on the shaft off-center from its axis. In an alternative embodiment of the invention, when the head is mounted in the sleeve, it is off-center from the sleeve axis and the sleeve is rotated.

In an exemplary embodiment of the invention, reaming is provided by eccentric rotation of the head. In an exemplary embodiment of the invention, the same head is used for both reaming and cutting by providing eccentric rotation of the head relative to the bone for reaming and concentric rotation for drilling.

In an exemplary embodiment of the invention, the cutting head of the hole forming apparatus is adapted for both reaming and drilling. In one example, the drilling function is supported by forward pointing sharp teeth. Optionally, the reaming function is supported by providing teeth that are sharp along an axial extent of the cutting head. Optionally, the fluting of the head is selected to support waste (bone) removal during reaming, for example, by providing a lower pointing angle than for drilling. Optionally, a section of the head adapted for cutting bone is relatively short, for example of the dimensions of a desired expanded diameter section.

In an exemplary embodiment of the invention, the reaming function is provided using at least one axially elongated fin that is sharpened in a rotation direction of the reaming. In an embodiment where opposite rotation directions selectively support reaming and cutting, a sharpened edge is optionally provided for the opposite rotation direction, for drilling. Optionally, the fins are axial and do not curve.

In an exemplary embodiment of the invention, a hole enlarging head is provided in which the hole enlarging head is expandable or distortable between a narrow diameter mode and a wider diameter mode. Such an enlarging head optionally includes drilling teeth thereon. Optionally, the hole enlarging head reams bone when enlarged. Alternatively or additionally, the hole enlarging head compacts bone when enlarged.

An aspect of some embodiments of the invention relates to treating a graft for fixation by increasing a thickness of an end thereof and/or by attaching an anchoring aid thereto. In an exemplary embodiment of the invention, the graft end is folded over and optionally sutured. Alternatively or additionally an anchoring aid device is attached to the end. Optionally, the anchoring aid is used for anchoring on its own, for example being resilient and/or otherwise expandable.

In an exemplary embodiment of the invention, the anchoring aid is formed on the end of the graft, for example by applying a setting material thereto. Optionally, the material is applied using a mold.

There is thus provided in accordance with an exemplary embodiment of the invention a device for forming expansion regions in a tunnel having a given lateral dimension in a bone, comprising:

an elongate body having and axis and adapted to fit through the tunnel;

a head, provided on said body and having outer dimensions not extending tans-axially to said body by an amount that would cause it to substantially impact the tunnel diameter in a first configuration and having an outer dimension that does impact the tunnel diameter in a second configuration;

wherein said head is rotatable and wherein said head is adapted to widen said tunnel in said bone by at least 5% in conjunction with said rotating. Optionally, said head is mounted at a distal end of said body. Alternatively or additionally, said head is adapted to rotate eccentrically relative to said axis, when said body is rotated.

In an exemplary embodiment of the invention, the device comprises a sleeve in which said body is mounted. Optionally, said head is mounted eccentrically relative to an axis of said sleeve. Alternatively said head is mounted non-eccentrically relative to an axis of said sleeve.

In an exemplary embodiment of the invention, said head is rotationally locked to said sleeve. Alternatively or additionally, said head has a rotationally limited motion relative to said sleeve, between 10 and 350 degrees of rotation. Alternatively or additionally, the device comprises an interlock which selectively rotationally locks said body to said sleeve. Optionally, said interlock includes a plurality of selectable interlocking settings. Alternatively or additionally, said interlock is directionally selective in at least two positions thereof preventing relative rotational motion one only in one rotation direction and one only in an opposite rotation direction.

In an exemplary embodiment of the invention, said sleeve has a lumen sized to receive said body and wherein said lumen is off-axis of an axis of said sleeve.

In an exemplary embodiment of the invention, said head is adapted to rotate centrically relative to said axis, when said body is rotated. Optionally, the device comprises a sleeve, wherein said body is mounted in said sleeve. Optionally, said head is mounted eccentrically relative to an axis of said sleeve.

In an exemplary embodiment of the invention, said head is radially enlargeable. Optionally, said head is inflatable.

In an exemplary embodiment of the invention, said head is adapted to crush cancellous bone in said tunnel to cause said widening.

In an exemplary embodiment of the invention, said head is adapted to ream cancellous bone in said tunnel to cause said widening.

In an exemplary embodiment of the invention, said head is adapted to remove cancellous bone in said tunnel to cause said widening.

In an exemplary embodiment of the invention, said head is adapted to widen said tunnel without moving axially.

In an exemplary embodiment of the invention, said device includes a drilling section adapted to form said tunnel. Optionally, said head includes said drilling section. Alternatively or additionally, said head is adapted to selectively drill or widen depending on a rotation direction of said head. Alternatively or additionally, said head is adapted to selectively drill or widen depending on an eccentricity of rotation of said head. Alternatively or additionally, said head is adapted to selectively drill or widen depending on a radial enlargement of said head. Alternatively or additionally, said head comprises at least one forward pointing drill edge. Alternatively or additionally, said head comprises at least one axially elongated edge adapted to cut bone for drilling.

In an exemplary embodiment of the invention, the device comprises a plurality of depth indicating axial markings.

In an exemplary embodiment of the invention, the device comprises an axially positionable depth limiter.

In an exemplary embodiment of the invention, said head and said body are cannulated.

There is also provided in accordance with an exemplary embodiment of the invention, a head adapted for drilling a tunnel and for widening an existing tunnel, comprising:

(a) at least one drill cutting edge arranged for cutting bone in a forward pointing direction; and

(b) at least one bone cutting edge arranged for cutting the bone transverse to the forward pointing direction and arranged in a general axial direction. Optionally, the head comprises at least a second substantially axially arranged bone cutting edge for adapted for cutting at an opposite rotation direction of said head than a rotation direction for which said head drills said tunnel. Alternatively or additionally, said head is mounted off-center on an elongate rod.

In an exemplary embodiment of the invention, said head is cannulated.

There is also provided in accordance with an exemplary embodiment of the invention a method of fixating soft material to bone, comprising:

(a) forming a tunnel in said bone;

(b) forming an expansion region in at least a part of said tunnel;

(c) inserting soft material into said expansion region via said tunnel; and

(d) fixating said soft tissue in said tunnel. Optionally, said soft material comprises a ligament or a tendon or a ligament or a tendon replacement graft. Optionally, said fixating is mechanical attachment. Optionally, said mechanical attachment is a mechanical interference attachment. Optionally, said mechanical attachment is a one way attachment prevent motion in only one direction along said tunnel.

In an exemplary embodiment of the invention, said mechanical attachment uses an expanding element to lodge in said expansion. Alternatively or additionally, said mechanical attachment uses a non-expanding element to lodge in said expansion. Alternatively or additionally, said mechanical attachment is adapted to hold on its own for less than 2 months. Alternatively or additionally, said mechanical attachment bio-degrades. Alternatively or additionally, said mechanical attachment uses a biodegradable element.

In an exemplary embodiment of the invention, said mechanical attachment does not use a setting element.

In an exemplary embodiment of the invention, said fixating does not include adhesive fixating.

In an exemplary embodiment of the invention, said fixating comprises adhesive fixating. Optionally, said fixating comprises providing a settable material in said expansion.

In an exemplary embodiment of the invention, forming an expansion comprises crushing bone adjacent said tunnel.

In an exemplary embodiment of the invention, forming an expansion comprises removing bone adjacent said tunnel.

In an exemplary embodiment of the invention, forming a tunnel and forming an expansion comprise using a same tool for drilling and expansion. Optionally, the method comprises changing a mode of said tool by changing a rotation direction thereof.

In an exemplary embodiment of the invention, said expansion region is tapered towards said tunnel.

In an exemplary embodiment of the invention, said tunnel extends in two directions away from said expansion region.

In an exemplary embodiment of the invention, inserting soft material comprises pushing said soft material into said expansion region.

In an exemplary embodiment of the invention, inserting soft material comprises pulling said soft material into said expansion region.

In an exemplary embodiment of the invention, the method comprises pre-treating said material to assist fixating thereof. Optionally, pre-treating comprises thickening using a suture. Alternatively or additionally, pre-treating comprises thickening using a setting material.

In an exemplary embodiment of the invention, the method comprises attaching an end of said soft material to a second bone.

In an exemplary embodiment of the invention, said ligament is an anterior cruciate ligament.

In an exemplary embodiment of the invention, said ligament is a posterior cruciate ligament.

In an exemplary embodiment of the invention, said ligament is a shoulder ligament.

There is also provided in accordance with an exemplary embodiment of the invention, a method of pre-treating graft material to facilitate fixating to bone, comprising:

(a) providing a graft material suitable to be used for a ligament or tendon; and

(b) increasing a cross-section of said graft at at least one end thereof. Optionally, increasing a cross-section comprises attaching an anchoring aid to said graft. Optionally, said anchoring aid comprises a layer of a settable material said graft.

In an exemplary embodiment of the invention, increasing a cross-section comprises folding said graft.

In an exemplary embodiment of the invention, increasing a cross-section comprises tying said end with a suture.

In an exemplary embodiment of the invention, the method comprises adding a suture loop for pulling to said graft.

There is also provided in accordance with a exemplary embodiment of the invention, an enhanced graft formed as described herein.

### BRIEF DESCRIPTION OF FIGURES

Particular exemplary embodiments of the invention will be described with reference to the following description of embodiments in conjunction with the figures, wherein identical structures, elements or parts which appear in more than one figure are generally labeled with a same or similar number in all the figures in which they appear, in which:

FIG. 1 is a schematic illustration of a left knee joint in flexion prepared for reconstruction according to an exemplary embodiment of the invention;

FIG. 2 is a flow diagram of a method of replacing a damaged ligament, according to an exemplary embodiment of the invention;

FIG. 3 is a schematic illustration of an expansion device, according to an exemplary embodiment of the invention;

FIG. 4A is a schematic illustration of an alternative expansion device, according to an exemplary embodiment of the invention;

FIG. 4B is a schematic illustration of an alternative expansion device, according to an exemplary embodiment of the invention;

FIG. 5 is a schematic illustration of an alternative expansion device, according to an exemplary embodiment of the invention;

FIG. 6A is a schematic illustration of an alternative expansion device before deployment, according to an exemplary embodiment of the invention;

FIG. 6B is a schematic illustration of the expansion device of FIG. 6A after deployment;

FIG. 7A is a schematic illustration of a left knee joint in flexion with a graft inserter deployed, according to an exemplary embodiment of the invention;

FIG. 7B is a schematic illustration of a magnified view of the distal end of the graft inserter of FIG. 7A;

FIG. 8A is a schematic illustration of a graft mounted on a graft inserter, according to an exemplary embodiment of the invention;

FIG. 8B is a schematic illustration of a graft mounted on a graft inserter, according to an alternative exemplary embodiment of the invention;

FIG. 8C is a schematic illustration of a graft mounted on a graft inserter with a syringe deployed for injecting fixation material, according to an exemplary embodiment of the invention;

FIGS. 9A-9E are schematic illustrations of a graft with anchoring at its distal end, according to exemplary embodiments of the invention;

FIGS. 10A-10D are schematic illustration of geometries of expansion regions in a bone, according to an exemplary embodiment of the invention;

FIGS. 10E and 10F illustrate the fixation of a graft in an expansion region using fixation material, in accordance with an exemplary embodiment of the invention;

FIG. 11A is a schematic illustration of an outer sleeve of a drill and void expansion device, according to an exemplary embodiment of the invention;

FIG. 11B is a schematic illustration of an inner rod of a drill and void expansion device, according to an exemplary embodiment of the invention;

FIG. 11C is a schematic illustration of an assembled drill and void expansion device using the elements of FIGS. 11A and 11B, according to an exemplary embodiment of the invention;

FIGS. 11D and 11E are partial cut away side and cross-sectional views, respectively of the assembled device of FIG. 11C, in accordance with an exemplary embodiment of the invention;

FIG. 12A is a schematic illustration of an alternative assembled drill and void expansion device positioned for expansion, according to an exemplary embodiment of the invention, in a reaming mode;

FIG. 12B is a schematic illustration of the assembled drill and void expansion device of FIG. 12A positioned for drilling, according to an exemplary embodiment of the invention; and

FIG. 13 is a schematic illustration of an assembled drill and void expansion device with a depth control mechanism, according to an exemplary embodiment of the invention;

### DETAILED DESCRIPTION OF EMBODIMENTS

General Description

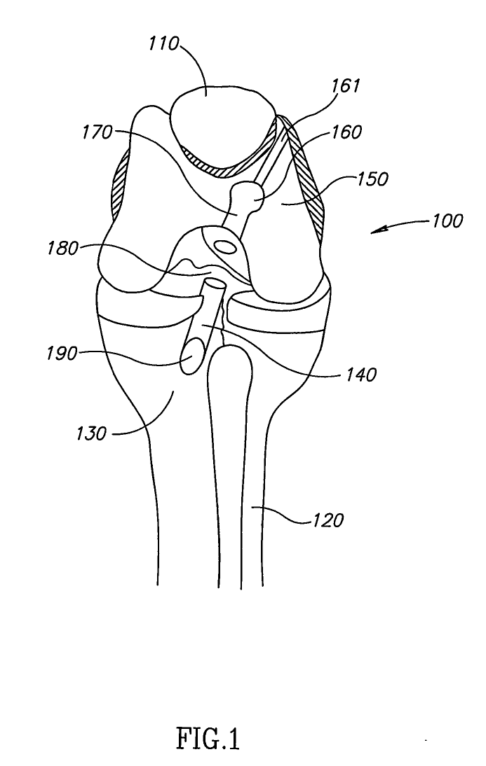

FIG. 1 is a schematic illustration of a left knee joint 100 in flexion prepared for replacement of a damaged ligament according to an exemplary embodiment of the invention. FIG. 1 shows the proximal ends of a tibia 130 and a fibula 120, meeting a femur distal end 150. A patella 110 is shown positioned above the femur distal end 150.

In an exemplary embodiment of the invention, a tunnel 190 is prepared comprising a through hole 140 through the proximal tibia, continuing through a joint void 180 between the bones, to a hole 170 in the distal femur 150. In an exemplary embodiment of the invention, the diameter of a distal end of hole 170 in the distal femur end 150 is enlarged to form an expansion region 160 for anchoring a ligament replacement as described below. Optionally, tunnel 190 is planned to be located approximately between the end attachment points of the original ACL. In an exemplary embodiment of the invention, a replacement ligament is inserted through tunnel 190 and hole 170 (following removal of the damaged ACL) and attached at each end to the respective bones as a replacement for a damaged ACL.

Optionally, a hole 161 is formed which extends past expansion region 160. Optionally, this hole is formed using a pin and/or a K-wire. Optionally, the ligament is pulled through hole 170 into expansion region 160 via hole 161 which optionally extends out of femur 150.

Optionally, fixation material may be injected via one or more of hole 161, a separate dedicated hole, or via hole 170. Optionally, the fixation material is injected when it is already partly set, possibly reducing leakage. Alternatively, a two component fixation material is used and the two components, for example a resin and a hardener, are injected separately, optionally via separate access holes, to expansion region 160. Alternatively or additionally, one or both components (or only one if only one is used) may be provided on the graft ligament itself, for example as a coating or in a capsule.

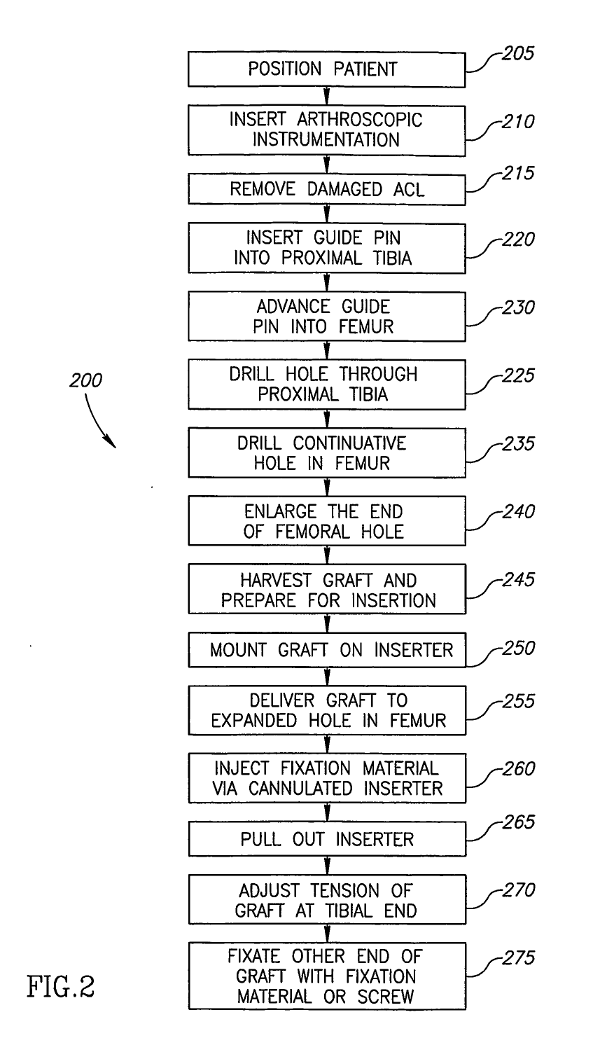

FIG. 2 is a flow diagram 200 of a method of replacing/reconstructing a damaged ligament, according to an exemplary embodiment of the invention. In an exemplary embodiment of the invention, a patient is positioned (205) for operation depending on the ligament that needs to be repaired. While this description herein focuses on the replacement of an ACL, the method described can be implemented in replacement of other ligaments as well. In replacing an ACL the patient's knee is generally bent in flexion to form an angle of approximately 90° between the femur and the tibia.

Once the patient is positioned (205), an incision is made and arthroscopic instrumentation is optionally inserted (210) to internally view the exact positioning of the damaged ligament As commonly practiced, the arthroscopic instrumentation is used to cut the ends of the damaged ligament and remove (215) it. In an exemplary embodiment of the invention, guide wire or K-wire is inserted (220) into the proximal end 130 of the tibia in order to form a thin guide path in the bone. When the guide wire is correctly placed, for example based on considerations known in the art, the guide wire is advanced toward and through femur end 150, optionally in a location close as possible to the original location of the ACL. Optionally, the guide wire is further advanced forming hole 161. Optionally, a standard bone drill (optionally cannulated to ride on the guide wire) is used to expand the guide path (230) to form hole 140 and then hole 170 of a desired diameter (for example to fit a replacement ligament). Typically the hole is directed to exit proximal tibia 130 and enter joint void 180 as near as possible to the original attachment position of the ACL.

In some cases, drilling and advancing of the guide wire are interleaved. First the proximal tibia 130 is drilled to form hole 140 and then the guide wire is advanced into femur 150 (225). Then hole 170 is drilled (235) in femur 150.

In an exemplary embodiment of the invention, the diameter of tunnel 190 is between 5 or 6 mm and 10 mm. Other methods as known in the art, for accessing the joint to perform the procedure, may be practiced, for example using an open incision rather than an arthroscopic procedure.

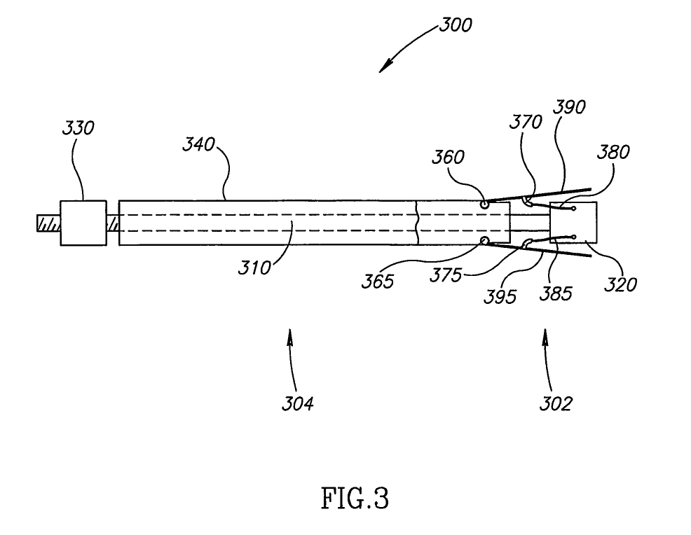

In an exemplary embodiment of the invention, expansion region 160 at the distal end of femora hole 170 is created (240) using an expansion device. An example of such an expansion device is an expandable balloon (optionally biodegradable) which is inflated with liquid to crush cancellous bone. Alternatively or additionally, other devices are used, for example a cannulated expandable drill, an expandable reamer, an eccentric reamer or eroder or an expandable polymer tube. Examples of suitable devices are shown in FIGS. 3-6 and 11-13 and described below. Optionally, these devices prepare expansion region 160 to have a diameter of between 0.5 mm to 7 mm larger than a cross-sectional diameter of hole 170.

In an exemplary embodiment of the invention, expansion region 160 is prepared such that a solid having a geometry and size of expansion region 160 would be anchored in place so it cannot be removed through hole 170. In some cases, the geometry is selected so that even a solid with a diameter smaller than that of expansion region 160, but greater than that of hole 170 would not be retractable. Optionally, expansion region 160 and/or hole 170 are not cylindrical. As will be described below, expansion and drilling may be performed without removing the drill, in some embodiments of the invention.

In an exemplary embodiment of the invention, hole 170 has a diameter between 6 and 10 mm; the K-wire has a diameter between 2.2 and 3.2 mm; expansion region 160 has a diameter between 7 and 15 mm and a length of between 10 and 15 mm, for example; and the degree of expansion can be 10%, 20%, 40%, 80%, 110% or more greater than the diameter of hole 170. Greater sizes may damage or weaken the bone more but support a better anchoring. These sizes are only exemplary and may depend on the exact procedure being performed.

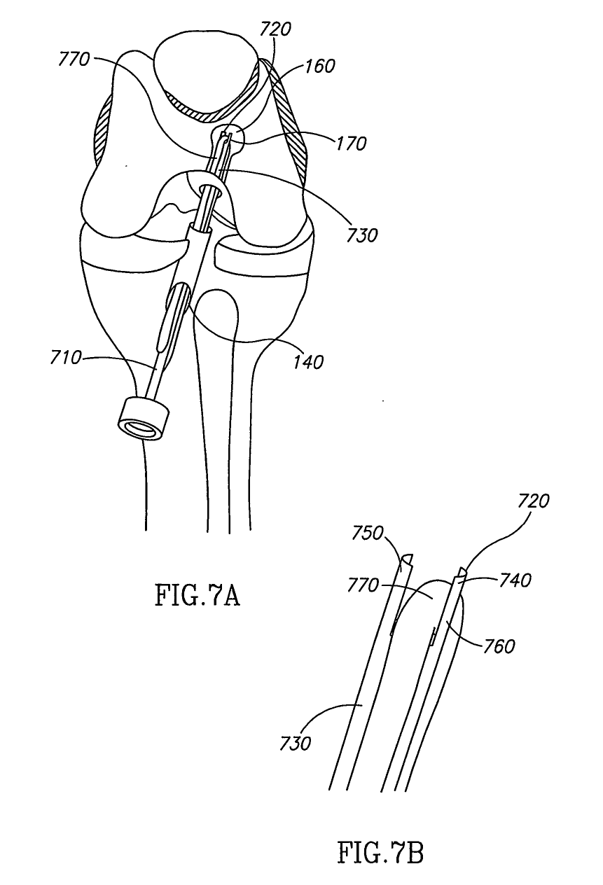

FIG. 7A is a schematic illustration of a left knee joint in flexion shown with a graft inserter deployed, according to an exemplary embodiment of the invention. FIG. 7A will be referenced to explain the rest of flow diagram 200. FIG. 7A shows a cannulated inserter 710 deployed into tunnel 190. In an exemplary embodiment of the invention, a graft 770 is harvested (245) and prepared for insertion into tunnel 190. For example a tendon graft, with or without a piece of bone at one or both ends, may be taken from the patient's patellar tendon or hamstring tendon (semitendinosus tendon, with or without gracilis tendon) in order to replace the ACL. In an exemplary embodiment of the invention, graft 770 is mounted (250) over a distal end 720 of cannulated inserter 710 in order to deliver (255) an end of graft 770 to expansion region 160 at the distal end of tunnel 190.

In some embodiments of the invention, cannulated inserter 710 comprises a cannulated shaft 730. In an exemplary embodiment of the invention, a fixation material, for example a bone void filler such as calcium phosphate (e.g., Biocement, Calcibone by Biomet-Merck) or calcium sulfate (e.g. Osteo-V or by Central Medical Technologies Inc.), is introduced, optionally through the cannulated inserter, to anchor the ligament replacement. Alternatively or additionally, a bone cement such as Polymethyl Methacrylate (PMMA) is used. In an exemplary embodiment of the invention, the fixation material is of a spongy nature, for example a spongy substance like alga which expands when brought in contact with a humid environment. Optionally, the fixation material is biodegradable and optionally encourages bone ingrowth.

Optionally, an expandable element is used for fixation, for example a fluid or cement filled balloon, a rubber plug or a self-expanding shape memory or super elastic element. In an alternative embodiment, a device (e.g., similar to an expandable inter-vertebral cage) such as described in PCT/IL00/00058 filed on Jan. 27, 2000, now published as WO 00/44319, the disclosure of which is incorporated herein by reference, is used. Optionally, this device is formed of a plastic material, for example, a silicone or polyurethane polymer. Optionally, the expandable element is used instead of or, optionally, in addition to fixation material. This device comprises, for example, a slotted tube which as it is axially compressed, pairs of axial slots define sections of the tube which extend away from the tube surface.

It should be noted that once bone ingrowth occurs, the durability of the element used to assist fixation/anchoring may be less important and a biodegradable element may be desirable to minimize foreign material in the body. Optionally, the element is only capable of maintaining the fixation on its own for three or two months or less, for example, 1 month or 2 weeks.

In an exemplary embodiment of the invention, the fixation materiel is injected (260) or otherwise inserted into expansion region 160 by way of cannulated shaft 730. In an exemplary embodiment of the invention, between 1 cc to 6 cc is used to form a plug in expansion region 160 or to fill expansion region 160 and anchor graft 770. Optionally, the fixation material is viscous enough to allow it to harden in the position it is inserted without leaking out before hardening. In an exemplary embodiment of the invention, bone growth, stimulating factors, anti-biotic materials, anti-inflammatory materials and/or other bio-active materials are added to the fixation material to provide, for example, for more rapid bone growth. The actual materials and amounts depend, for example, on the mechanical effect to be achieved. In some cases, the fixation material serves to strengthen the bone matrix (e.g., cancellous bone).

In an exemplary embodiment of the invention, the fixation material serves as a plug which has a diameter greater than that of hole 170, thus preventing its retraction (e.g., by mechanical interference). Optionally, the plug is adhered to or otherwise attached to expansion region 160 or the surrounding bone. Optionally, hole 170 is left free of the fixation material. Alternatively, a different fixation material, such as bone chips may be placed in hole 170, optionally serving for filling rather than fixation. Alternatively, the plug extends through hole 170, optionally to the surface of the bone. Optionally, underfilling is desired, to prevent leakage of the filing material.

In an alternative embodiment of the invention, fixation material is provided in another manner. In one exemplary embodiment, a narrow diameter hole is formed to expansion region 160, for purpose of injecting cement or other fixation material into it. Such a hole may be drilled from a different direction than hole 170, for example perpendicular thereto. Optionally, the hole is punched by an injecting needle. Optionally, the additional hole is an axial extension of hole 170 (e.g., hole 161), albeit at a same or reduced diameter and not at the diameter of expansion region 160. Optionally, a K-wire is used to form that extension of hole 170, which is optionally expanded, for example by pushing a tube along the K-wire or by advancing a bone drill, bone punch or bone compactor along the K-wire.

Optionally, fixation material is provided before inserting the graft. Optionally, the fixation material is provided inside a capsule which dissolves after a time or crushed upon graft insertion, releasing the fixation material. Optionally, the graft is pre-coated with a layer of semi-set or completely set material, such as bone cement.

After positioning of graft 770 and optionally after injection of the fixation material, inserter 710 is pulled out (265) leaving graft 710 inserted. Optionally, after a short delay, for example 10 to 20 minutes, depending on the fixation material used, the material hardens enough to allow exertion of tension on the graft from the tibia end of the graft, in order to adjust (270) the position of the replacement ligament at the proximal tibia side. In an exemplary embodiment of the invention, the graft at the tibial side is fixated (275) by injecting fixation material or a different adhesive material in tibia hole 140. Optionally an expansion based fixation is used at the tibia side in addition to or instead of at the femur side. Alternatively, the tibia side of the graft is fixated using a standard fixation method, for example a screw.

Other tendons and ligaments may be attached using the methods described herein. In one example, a rotator cuff is connected using the method by creating a void in a bone and anchoring a ligament in the void. Optionally, this method is also used for attaching a ligament extension which is attached to a torn ligament (or tendon), to a bone. Optionally, hole 170 is formed along or at an original attachment point for example as part of a cleaning step.

Other soft material may be fixated/anchored using the methods described herein. In one example, pelvic floor fixation and support, bladder suspension, urethral support (e.g., for incontinence treatment) or urethral support are provided using a sling or material (also a ligament may be used) which is anchored to bone (e.g., the pelvic or pubic bones) using methods described herein. A potential advantage of these types of fixation is that the fixation point is not felt during sexual intercourse or other daily activities. In an exemplary embodiment of the invention, this type of fixation is used for cystourethropexy. Also other procedures where soft tissue is supported by a sling attached to bone may use the methods described herein, for example, support of tissue sin the air passages.

Void Expansion Device

FIG. 3 is a schematic illustration of a void expansion device 300, according to an exemplary embodiment of the invention. In an exemplary embodiment of the invention, femoral hole 170 is initially prepared with a constant diameter along its length, for example using a drill as well known in the art. Optionally, void expansion device 300 includes a drill bit at a head 320 thereof, which is used to form the hole.

In an exemplary embodiment of the invention device 300 comprises a relatively uniform diameter shaft section 304 and a variable diameter head section 302. After insertion and/or drilling, during which head section 302 has a same or smaller diameter than shaft section 304, head section 302 is expanded to have a greater diameter. When device 300 is rotated, the head section will ream out material from the bone and create or expand expansion region 160. In some embodiments, the expansion will create the void even without (or with minimal) rotation, for example by compression of the bone material. However, this may depend on the hardness of the bone. In an exemplary embodiment of the invention, the diameter at the expansion is greater than that of hole 170 by between 0.5 mm to 7 mm in order to provide sufficient interference to anchor graft 770.

Optionally, rotation of device 300 is using a manual handle (not shown). Alternatively, a motor, for example an electric or pneumatic motor is used for rotation. In manual rotation, a gear or other system for providing mechanical advantage and force modification may be used.

In an exemplary embodiment of the invention, device 300 comprises an inner rod 310 encased in an over-sleeve 340. Head section 302 comprises one (or more) blades (390, 395) coupled to the side of over-sleeve 340 by a hinge (360, 365). Optionally, each blade (390, 395) has a pin (370, 375 respectively) extending inward toward inner rod 310. Optionally, head 320 is positioned on top of inner rod 310, with two struts (380, 385) extending downward toward the pins (370, 375).

In an exemplary embodiment of the invention, device 300 is inserted into tunnel 190 with blades 390 and 395 lying substantially parallel to over-sleeve 340. Optionally, if head 320 is pulled downward, struts (380, 385) push on pins (370, 375) and blades (390, 395) push outward. By rotating inner rod 310 while pulling down on head 320 toward over-sleeve 340, device 300 causes blades (390, 395) to push out and cut away from the walls of tunnel 190 at a distal end thereof to form expansion region 160.

Other radial expansion mechanisms can be used as well. In one example, hinges 360 and 365 elastically urge blades 390 and 395 away from head 320. Struts 380 and 385 serve to selectively counteract the elastic urging. In another example, head 320 is a cone with its narrow end pointing proximally, so that retraction of head 320 causes blades (390, 395) to extend radially. Similarly, blades 390 and 395 can be formed to have an inside inclined surface that is urged away when head 320 retracts proximally.

In an exemplary embodiment of the invention, the proximal end of inner rod 310 comprises threading adapted to accommodate a nut 330. Optionally, rotating nut 330 clockwise or counter clockwise, moves inner rod 310 proximally or distally relative to over-sleeve 340 exerting axial force on head 320.

In some embodiments of the invention, rod 310 and/or sleeve 340 of device 300 are comprised from a flexible metal alloy, for example Nitinol, in order to provide flexibility and allow insertion in tunnel 190 even if the components of tunnel 190 (140, 180, 170) are not exactly aligned.

Device 300 optionally remains in place axially during reaming. Alternatively, it may be advanced (distally) or retracted (proximally). In embodiments where an expansion is to be formed in two bones, two expanding sections are optionally provided, one at the distal end as shown and another situated at a suitable more proximal location, for simultaneously creating expansion regions at two or more locations.

Expansion Region Geometries

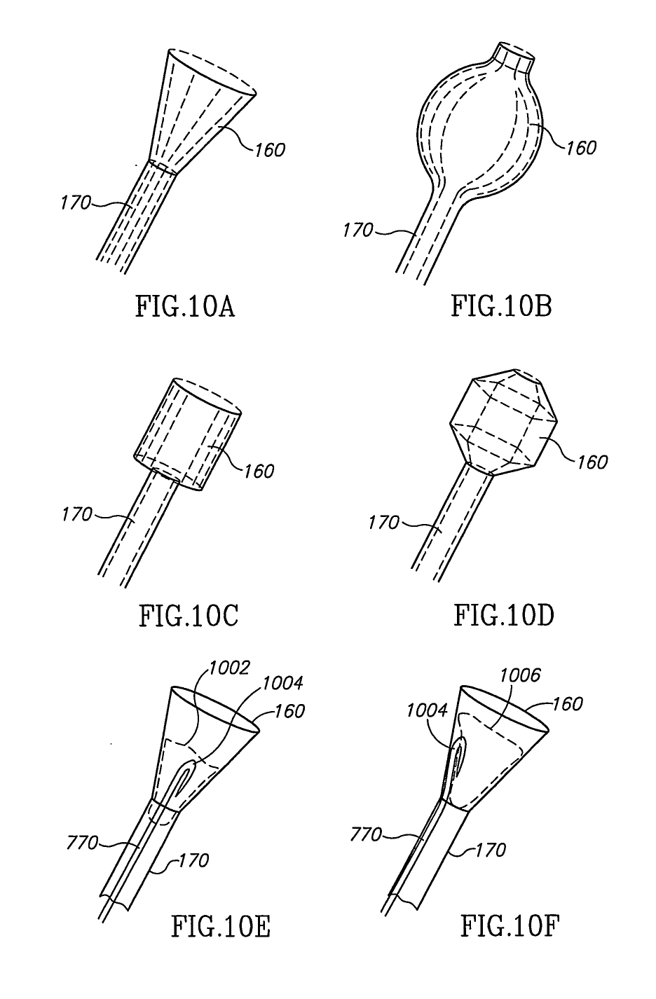

FIGS. 10A-10D are schematic illustrations of the geometry of expansion region 160 created by an expansion device, according to exemplary embodiments of the invention.

In an exemplary embodiment of the invention, device 300 forms an expansion region 160 as illustrated in FIG. 10A and described above. In some embodiments of the invention, a device that creates a symmetrical expansion is used. Alternatively, a device that creates an expansion that tapers wide to narrow or narrow to wide (e.g. device 300) is used. In some embodiments of the invention, the shape of the expansion is selected dependent on the type of bone being drilled in and the type of replacement ligament used. A potential advantage of an inclined shape as shown in FIG. 10A is that a plug of material can progressively wedge into hole 170 as the plug is retracted proximally towards hole 170.

FIG. 10B shows a smooth expansion, created for example by providing an inflatable balloon at a tip of device 300. A potential advantage is a lack of sharp edges which, if present may cause crumbling of the bone during and/or after fixation.

FIG. 10C shows a geometry with a step at the interface between expansion region 160 and hole 170. Optionally, this step serves to securely anchor the plug. Optionally, this step is achieved by blades 390 and 395 pointing proximally rather than distally. A similar mechanism as described above may be used to collapse and expand the blades. Optionally, blades 390 and 395 are mounted on a balloon, such that when the balloon is inflated, they extend out of device 300 and remain parallel to its axis.

FIG. 10D shows a geometry including inclined sections at either end of expansion region 160. This geometry may be useful to prevent distal migration of the plug.

While the geometries shown are rotationally symmetric around the axis of hole 190, this is not essential. In some embodiments of the invention expansion region 160 is offset to one side, for example, for ease of creation. In some cases this may be desirable to avoid reaming cortical bone or to avoid other structures. In other cases, such a geometry may be mechanically useful.

It should also be noted that expansion region 160 need not be circular in cross-section. Or example, a triangular cross-section or an elliptical cross-section may be provided. The cross-section may be a function of the tool used for forming expansion region 160.

FIGS. 10E and 10F illustrate the fixation of graft 770 in expansion region 160 using fixation material (or a plug), in accordance with an exemplary embodiment of the invention.

FIG. 10E shows an example where the fixation material surrounds a folded (or otherwise thickened) end 1004 of graft 770, forming a plug 1002. This plug cannot retract out of hole 170 from geometrical considerations, and thus prevents graft 770 from retracting. In some embodiments plug 1002 fills expansion region 160. In some embodiments plug 1002 extends into (and possibly fills) holes 170 and/or 161. In some embodiments, plug 1002 fixates to the bone surrounding expansion region 160, for example if the plug is a glue plug. In other embodiments, for example if plug 1002 is a balloon or a rubber plug mounted on graft 770, this fixation to the bone may not occur. Optionally, (not shown) the distal end of graft 770 projects past plug 1002.

FIG. 10F shows an example where thickening 1004 is situated on a side of a plug 1006. Optionally, plug 1006 is attached to graft 770. Alternatively, plug 1006 and graft 770 not attached. As can be seen, due to friction, graft 770 cannot retract from expansion region 160, even if there is no adhesion between graft 770, plug 1006 and the bone surrounding expansion region 160. Optionally, plug 1006 is held mechanically by tension of graft 770. Alternatively or additionally, plug 1006 is held mechanically by engaging both hole 170 and expansion region 160. The various geometries of FIGS. 10A-10D may assist such engaging, as might the texture of the inner wall of expansion region 160. Optionally, plug 1006 does not fill all of expansion region 160, for example, filling only 80%, 50%, 40% or less, for example, to reduce the amount of foreign material in the body.

Expandable Reamer

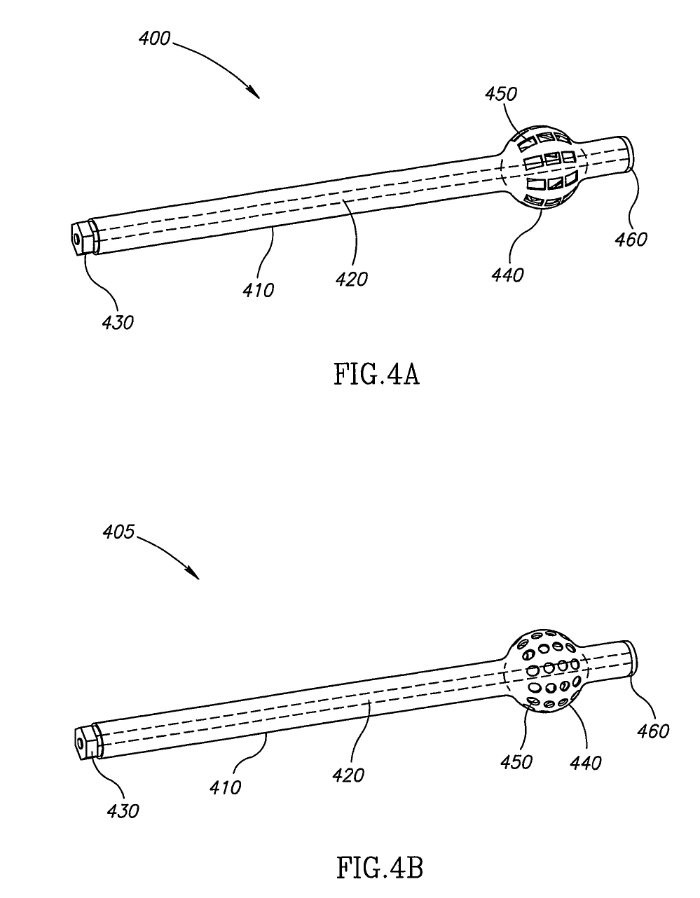

FIG. 4A is a schematic illustration of an expandable reamer 400, in accordance with an exemplary embodiment of the invention and which may be used for forming expansion region 160, for example in the geometry of FIG. 10B.

In an exemplary embodiment of the invention, reamer 400 comprises a cylindrical over-sleeve 410 with an inner rod 420. The distal end of reamer 400 comprises a cover 460, which couples between inner rod 420 and over-sleeve 410. Optionally, the proximal end of inner rod 420 comprises threading adapted to accommodate a nut 430. When tightening nut 430, force is exerted on over-sleeve 410. An expandable portion 440 is provided with a fenestrated architecture 450 to allow expansion. Optionally, sleeve 410 is weaker at expandable portion 440, for example being chemically or mechanically weakened or being thinner than the rest of sleeve 410. In an exemplary embodiment of the invention, during insertion and/or removal, sleeve 410 is not radially expanded.

In some embodiments of the invention, the expandable portion is adjacent to cover 460. Alternatively, the expandable portion is slightly distanced from cover 460 (e.g. 5 mm-15 mm), for example, to allow the expandable portion to expand better. As a result of the exerted force expandable portion 440 expands outward radially. After or during expansion, the reamer is rotated to remove bone and form expansion portion 160. Optionally while expanding, cancerous bone is crushed and the distal end of hole 170 in the distal femur is expanded to form expansion region 160 (see FIG. 10B). In an exemplary embodiment of the invention, by releasing nut 430, over-sleeve 410 regains the original cylindrical shape so that it can be removed from tunnel 190. In an alternative embodiment of the invention, sleeve 410 is naturally distorted and only axial application of force by rod 420 keeps it at a uniform diameter. When the axial force is released, the expandable portion expands to the desired shape.

Optionally, rod 420 is hollow and/or is replaced by a cable.

In FIG. 4A fenestrated architecture 450 is shown with rectangular openings. FIG. 4B shows an alternative reamer 405 having circular openings. Optionally, other shaped openings can be used to form expandable portion 440. The shape of the openings is optionally selected according a bone removal rate, with rectangular openings optionally having a greater removal rate, which may be useful if a combined reaming and crushing function is desired. A mixture of different shapes may be provided as well. Various amounts of reaming and crushing may be provided, for example depending on rotation speed, expansion speed and opening sizes and shapes.

Optionally, sleeve 410 includes cut-outs which select the geometry of expanded portion 440. For example, triangular cut-outs may be used to provide a sharp angle between expansion 440 and sleeve 410. U.S. Pat. No. 6,780,175, the disclosure of which is incorporated herein by reference, describes geometries which may be used for reaming.

In some embodiments of the invention, expandable portion 440 is provided with an abrasive exterior so that rotation of reamer 400 files the inner walls of tunnel 190. Optionally, the apertures are sharpened, for example chemically or mechanically.

Optionally, the expansion of expandable portion 440 is gradual and is optionally synchronized to rotation. In one example, nut 430 turns with the rotation and slowly tightens and expands sleeve 410. Optionally, this allows for more efficient reaming.

Eccentric eroder

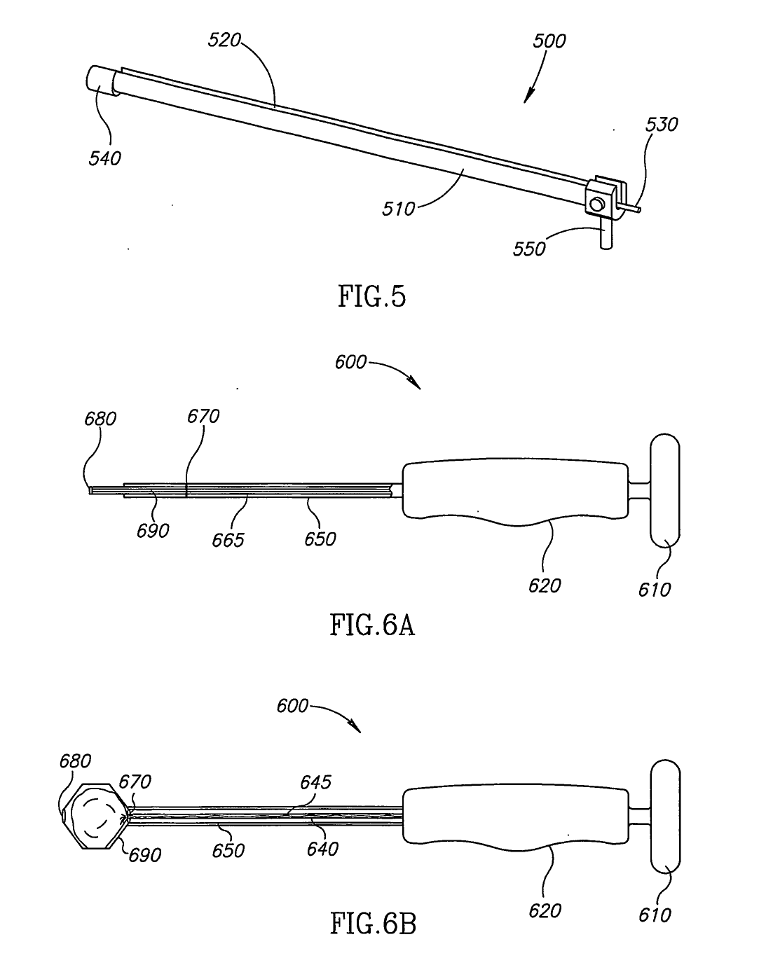

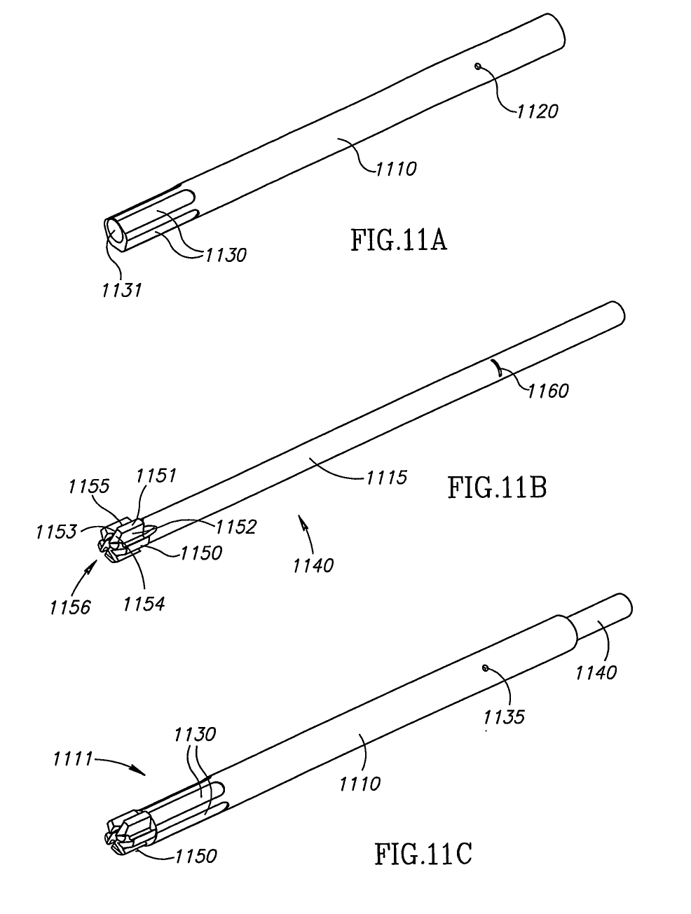

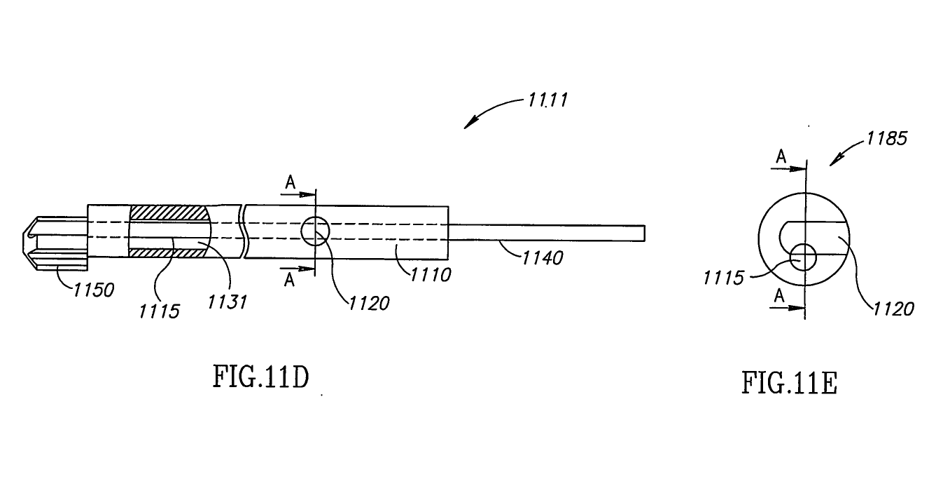

FIG. 5 is a schematic illustration of an eccentric eroding device 500, according to an exemplary embodiment of the invention. Eccentric eroder 500 comprises a delivery tube 510 with a groove 520 formed along it and an eroding head 540 having a shaft 530, to which head 540 is attached off-axis. While head 540 optionally includes one or more bone cutters, which for reasons of simplifying the drawings are not shown. A design similar to that in FIG. 11, may be used, for example.

When eroder 500 is inserted to tunnel 190, inner rod 530 is positioned in groove 520 so that cutting head 540 is centered relative to delivery tube 510. Once eroder 500 is in position in tunnel 190, inner rod 530 is rotated causing cutting head 540 to widen the hole at the distal end 170 of the femur. FIG. 10C illustrates expansion region 160 after being widened by eccentric eroder 500. In some embodiments of the invention, a handle 550 is connected to the proximal end of inner rod 530 in order to assist in rotation of inner rod 530.

Optionally, prior to rotation of rod 530, rod 530 is trans-axially positioned relative to the axis of tube 510, for example, by inserting an elongate insert into groove 520 to ensure that rod 530 does not move trans-axially.

It is noted that erosion or reaming may be limited to one angular sector of expansion region 160. Optionally, after one sector is eroded, tube 510 is rotated, for example, 90, 120 or 180 degrees, so that a more symmetric erosion of expansion region 160 is provided.

In an alternative embodiment of the invention, device 500 includes a channel rather than a groove. In use, head 540 is inserted into hole 190 and then over-sleeve 510 is inserted over it. Optionally, device 500 includes two channels, one on-axis and one off-axis. When inserted over rod 530 to engage the on-axis channel, device 500 acts as a drill. When inserted over the off-axis channel, device 500 acts as a reamer (e.g., if head 540 is attached centered on the axis of rod 530).

Combined Drill and Reamer

FIGS. 11A-11E are schematic illustrations of components and an assembled drilling and reaming device 1111 according to an exemplary embodiment of the invention. Device 1111 can operate in two modes, a drilling mode, in which the relatively uniform bore hole 170 is created and as a reamer, in which expansion region 160 is formed in hole 170. In some embodiments, no drilling mode is provided.

FIG. 11A shows an outer sleeve 1110 including one or more optional scalloped sections 1130, for assisting in bone removal. An aperture 1120 is optionally provided for locking to an inner rod, as described below. An off-center lumen 1131 is provided in sleeve 1110. The function of the lumen is described below.

FIG. 11B shows a drilling section 1140 including a reamer head 1150 mounted on a rod 11 15, with an optional hole or notch 1160 to match optional hole 1120. Optionally, head 1150 is mounted off-axis of rod 1115.

FIG. 11C shows rod 1115 mounted in sleeve lumen 1131 of sleeve 1110, with FIG. 11D being a side partially see-through view and FIG. 11E being a cross-sectional view 1185, in an embodiment of the invention where rod 1115 has a diameter smaller than an inner diameter of lumen 1131. In other embodiments, the rod diameter is substantially the same as the lumen diameter.

In one operational mode, rod 1115 is oriented in lumen 1131 so that head 1150 is centered relative to the axis of sleeve 1110. In this configuration, rotation of sleeve 1110 will provide a drilling action. Optionally, a pin 1135 is used to lock rod 1115 to sleeve 1110 via holes 1120 and 1160.

In another operational mode, rod 1115 (rather than sleeve 1110) is directly rotated and pin 1135 is disengaged. If sleeve 1110 is engaged in hole 190, this results in reaming action of head 1150, by virtue of head 1150 rotating off-axis (eccentrically). This may produce an expansion portion 160 on only one side of hole 190. In an alternative embodiment, one different locking hole is used to lock rod 1115 to sleeve 1110 so that head 1150 is centered and another hole is used so that rod 1115 and sleeve 1110 are locked to make head 1150 off-center. Thus, depending on the pin hole used, rotation of sleeve 1110 will result selectively in drilling or reaming.

Optionally more than one pin hole is prepared on inner rod 1115 and/or outer sleeve 1110 to allow selection of different levels of off-centered protrusion of drill head 1150 relative to outer sleeve 1110.

Optionally, pin 1135 is used for automatic conversion between a drill mode and a reaming mode. When sleeve 1110 is inserted deep enough into the bone pin 1135 contacts the outside surface of the bone, which as pressure is increased pushes against pin 1135 and rotates it out of hole 1160 (or into hole 160, depending on the embodiment), thereby selectively unlocking (or locking) sleeve 1110 and rod 1115 and changing the mode of operation.

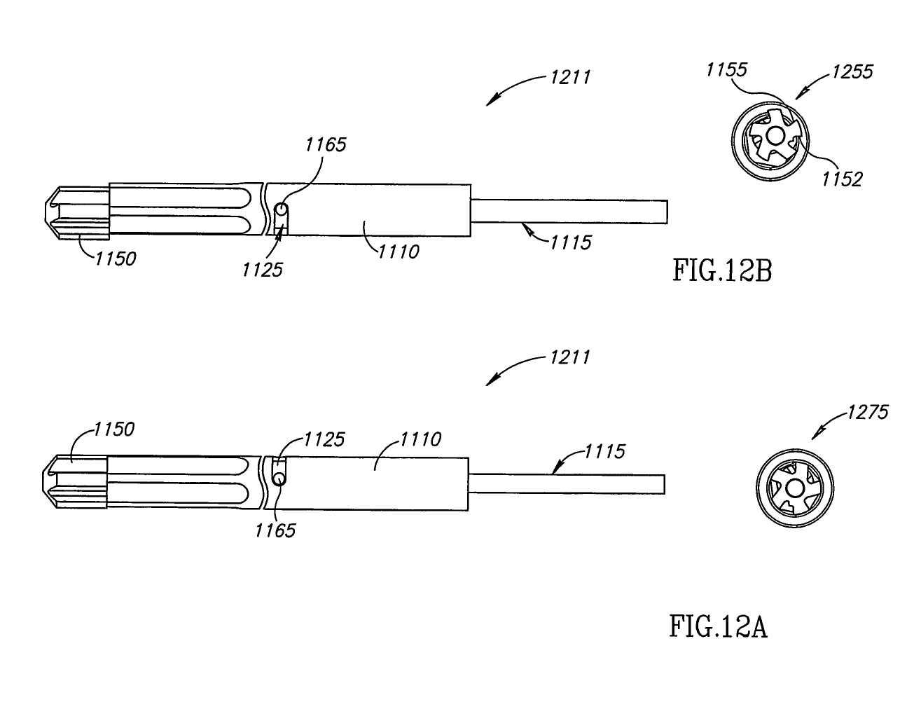

FIGS. 12A and 12B show an alternative embodiment of a drilling and reaming device 1211, with optionally which can function as a two mode drill/reamer.

Instead of a pin that locks outer sleeve 1110 to inner rod 1115, a projection 1165 extends from rod 1115 and rides in a circumferential slot 1125 in sleeve 1110. A similar mechanism may be provided by a protrusion or pin extending inwards from sleeve 1110 to lie in a slot in rod 1115.

When sleeve 1110 is rotated counter-clockwise with respect to rod 1115, as shown in FIG. 12B, rod 1115 is engaged in a position where head 1150 is off-center, providing a reaming function. This is also shown by reference 1255, a front view. Depending on the embodiment, either the sleeve and/or the rod are rotated.

When sleeve 1110 is rotated clockwise with respect to rod 1115, as shown in FIG. 12A, rod 1115 can rotate to a position where head 1150 is on-centered, providing a drilling function. This is also shown by reference 1275, a front view. Optionally, the edges of head 1150 are designed to support each function in a different rotation direction.

Alternatively to automatic (e.g., indirect) changing of function by manual changing of rotation direction, a knob may be provided to relatively rotate sleeve 1110 and rod 1115 and lock them in relative orientation, directly changing the function. Optionally, drilling or reaming is performed by rotating rod 1115.

In some embodiments of the invention, selection between drilling mode and expansion mode is done by displacement of inner rod 1115 relative to outer sleeve 1110 along the longitudinal axis. Optionally, a first position causes inner rod 1115 and outer sleeve 1110 to move together in alignment in order to drill. A second position lets inner rod 1115 move by itself with non-aligned motion in order to create expansion region 160. Optionally, this mechanism is provided by one or more elements (e.g., protrusions) on rod 1115 (not shown) engaging one or more inner elements (e.g., protrusions or notches) on sleeve 111 0.

In an exemplary embodiment of the invention, the K-Wire is removed before using void expanding devices in an eccentric rotation mode. Possibly, not removing the wire, for some embodiments, will prevent eccentric rotation relative to the K-Wire.

Depth Control

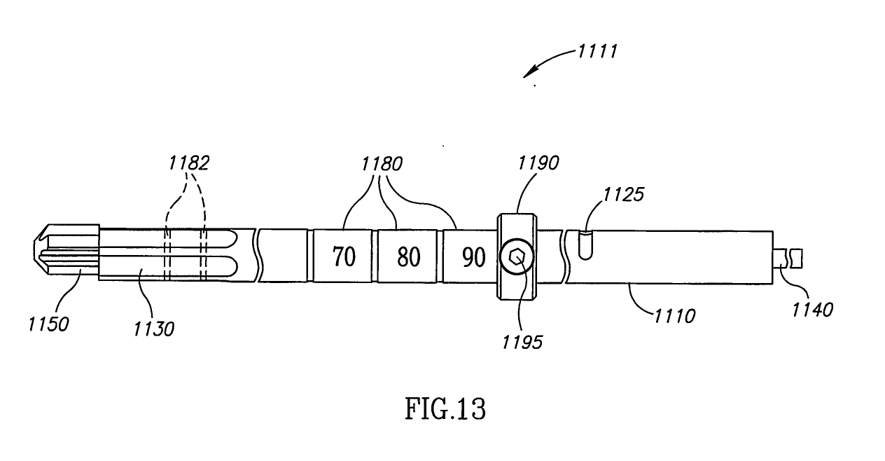

FIG. 13 shows two methods of depth control, each of which is optionally practiced in some embodiments of the invention. In one method, a plurality of depth markings 1180 indicates a depth of drilling. In another method, a lock 1190 with an optional locking nut 1195 prevent forward motion of the drill past a certain depth. Optionally, axial pressure against the lock causes (a) coupling, (or uncoupling or a change in coupling) between sleeve 1110 and rod 1115 or (b) movement of nut 330 or head 320 (FIG. 3), or (c) otherwise cause deployment of a reaming function. In this case, lock 1190 is optionally provided with a smooth surface and/or lubrication, to reduce damage to the bone at the entrance thereto.

Optionally, one or more depth markings 1182 are provided nearer a distal end of device 1111. Optionally, these markings are used during endoscopic procedures where imaging ability is provided inside the joint and near the bone (e.g., femur).

In some embodiments of the invention, inner rod 115 is cannulated to allow use of a K-wire and/or injection of glue or another fixation material.

Reaming and Drilling Head Design

Referring back to FIG. 11B, in an exemplary embodiment of the invention, head 1150 is designed to support both drilling and reaming. In an exemplary embodiment of the invention, head 1150 comprises one or more of fins 1151 which may be substantially axial or at a slight angle to the axis. Optionally, an angle of attack of 40° is used for the drilling. A reaming edge 1152 is optionally provided in the rotation direction used for reaming. For drilling, one or more of forward pointing bit tips 1153 are provided, each optionally provided with a drilling edge 1154. Optionally, the angle of attack of the reaming edges and/or of the drilling edges is 72°. Optionally, a hollow 1156 is defied between the drilling tips, for example to assist in tissue removal from the body.

If head 1150 operates in different modes for different rotations directions, a drilling edge 1155 may be provided on an opposite side of fins 1151. Optionally, only one reaming edge is provided, as shown in FIGS. 12A and 12B. Alternatively, more edges are provided. Optionally, no sharpened edge is provided for reaming.

Expansion Region Forming by Enlarging Element

FIGS. 6A and 6B show an expansion forming device 600 using a polymer tube which is expanded to an enlarged geometry, thereby forming an expansion region, optionally by compacting bone. FIG. 6A shows the tube before expansion and FIG. 6B, after expansion. Some variants of construction are shown in the two figures.

Referring to FIG. 6B, in an exemplary embodiment of the invention, polymer tube 690 is coupled to a tube 640 filled with a soft malleable material 645 such as silicone or polyurethane. An over tube 650 optionally prevents expansion along its length. Optionally, only a single tube is provided.

To expand tube 690, material 645 in tube 640 is advanced into tube 690. Optionally, tube 690 is short and is attached to overtube 650 at a ring 670. Optionally, material 645 in tube 640 is advanced by rotating a knob 610 relative to a body 620, so that the length of tube 640 is shortened. Alternatively, a syringe or other means may be used. Alternatively, knob 610 advances a plunger in tube 640. Alternatively, tube 640 itself is relatively soft and is pushed forward into tube 690.

Referring to FIG. 6A, optionally, an expandable device such as described in PCT/IL00/00058 filed on Jan. 27, 2000, now published as WO 00/44319, the disclosure of which is incorporated herein by reference, is used, in which a slotted tube is advanced against a stop to expand to a desired geometry. This is indicated in FIG. 6A, by a pushing tube 665 which pushes tube 690 against a stop 680 (which is optionally coupled to body 620, for example by an axial rod or cable). If the tube is of a soft material, for example between 60 Shore A and 60 Shore D, optionally no slots are provided. Optionally, a device such as described in PCT/IL2004/000527, filed on Jun. 17, 2004, the disclosure of which is incorporated herein by reference, is used. In this device, a slotted or unslotted polymer tube is axially compressed so that the tube radially expands. The device can be either cannulated or non-cannulated.

A potential advantage of compressing the bone rather than reaming it out is that better anchoring may be achieved.

While not stated explicitly for all embodiments, Optionally, the void expanding device (e.g., a balloon) may be cannulated and/or include a drill bit at the end.

Graft Insertion Devices

FIG. 7B is a schematic illustration of a magnified view of the distal end of a cannulated graft inserter 710, according to an exemplary embodiment of the invention. In an exemplary embodiment of the invention, the distal end 720 of graft inserter 710 is slotted to define two lateral plates 740, 750. Optionally, plates 740 and 750 are concave plates. Concave plates optionally fit the shape of tunnel 190 and/or maximize the thickness of the graft that can be inserted. In an exemplary embodiment of the invention, graft 770 is folded (at a point indicated by reference 760) over distal end 720 between plates 740 and 750 in order to strengthen graft 770 by using a double strand as the replacement ligament.

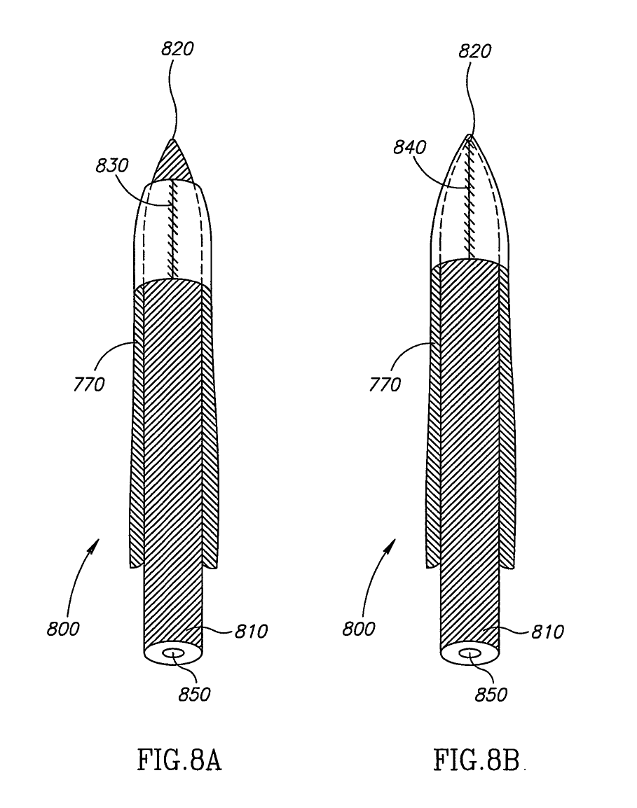

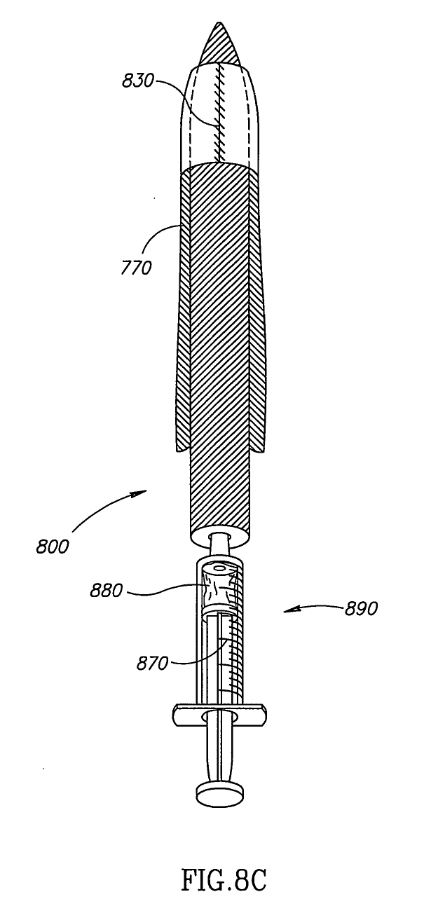

FIG. 8A is a schematic illustration of a graft 770 mounted on a graft inserter 800, according to an exemplary embodiment of the invention. In an exemplary embodiment of the invention, inserter 800 comprises a shaft 810 formed as a cylindrical tube that narrows at its distal end 820. In an exemplary embodiment of the invention, shaft 810 has a hollow tunnel 850 along its axis through the center. Optionally, fixation material is injected into tunnel 850 to distal end 820 (e.g., with distal end 820 being apertured) in order to deliver the fixation material to expansion region 160. In an exemplary embodiment of the invention a graft 770 is mounted like a cape around shaft 810. Optionally, the distal end of graft 770 is prepared to completely encircle shaft 810, for example two sides of graft 770 can be sutured 830 together near the narrow distal end of shaft 810 in order to encircle shaft 810 during insertion. Optionally, one or more grooves are defined along shaft 810, to accommodate the graft.

In some embodiments of the invention, shaft 810 is made from a rigid material, for example metal or a hard plastic. Optionally, shaft 810 is slightly flexible (e.g. nitinol), in order to overcome a slight displacement of the different sections comprising tunnel 190.

FIG. 8B is a schematic illustration of graft 770 mounted on graft inserter 800, according to an alternative exemplary embodiment of the invention. In this embodiment, graft 770 is sutured 840 so that the distal end 820 of shaft 810 is covered. Optionally, a small opening is left at distal end 820 to allow fixation material to pass from tunnel 850 to the exterior side of graft 770. Alternatively, this arrangement is used to assist fixation material to collect between graft 770 and the proximal side of expansion region 160.

Optionally, fixation material, such as an adhesive is affixed on the exterior side of graft 770 before its insertion.

FIG. 8C is a schematic illustration of graft 770 mounted on graft inserter 800 with a syringe 890 deployed in tunnel 850 for injecting fixation material 880, according to an exemplary embodiment of the invention. Optionally, syringe 890 comprises graduation marks 870, for controlling the quantity of fixation material injected to expansion region 160. Optionally, the amount of fixation material that fills the tunnel of shaft 810 is predetermined so that a user can calculate the amount exiting distal end 820. Optionally, the amount is estimated by visual inspection of leakage of material through the hole 170 by an endoscopic device.

Optionally, a soft rubber plug is used instead of a setting material or in addition to it, for example to prevent graft 770 or graft 770 attached to the fixation material, from retracting from expansion region 160. For example, a rubber (or similar deformable material) can be compressed and inserted into expansion region 160 to expand therein and anchor and/or prevent retraction of the graft. Optionally, the plug has a hardness of 50 Shore A. Optionally, the rubber biodegrades or includes a geometry or materials that promote ingrowth of tissue. Optionally, a stiffer plug, for example as described in U.S. Ser. No. 60/554,558, filed on Mar. 18, 2004, and PCT/IL2004/000527, filed on Jun. 17, 2004 or a biodegradable balloon as described in U.S. Ser. No. 60/534,377 filed on Jan. 6, 2004, the disclosures of which are incorporated herein by reference, is used.

It is noted that by preventing flexion beyond that of the procedure for a number of days or weeks until the plug (e.g., rubber or cement) completes anchoring, retraction of the ligament can be prevented even without adhesive fixation to the bone, if the ligament is generally under tension.

Graft Anchoring Aids

In an exemplary embodiment of the invention, various methods are used to secure or assist in securing an end of a ligament graft 770, in expansion region 160, optionally by increasing a thickness thereof or attaching an anchoring device thereto. Optionally, these methods are used also for cases where no expansion 160 is formed, for example, by the thickening r anchoring device engaging hole 170 by friction and/or adhesive.

In some embodiments of the invention, graft 770 is harvested with bone on at least one end. Optionally, the end with bone attached is inserted to expansion region 160 in order to enhance the anchoring of the graft on the side of the femur. Optionally, fixation material is added around the bone to form an anchor, which is wider than tunnel 190. In some embodiments of the invention, expansion region 160 is filled with enough fixation material to prevent the anchor from moving. Alternatively or additionally, the bone at the end of the graft is attached with adhesive to the inner walls of expansion region 160 to prevent it from moving.

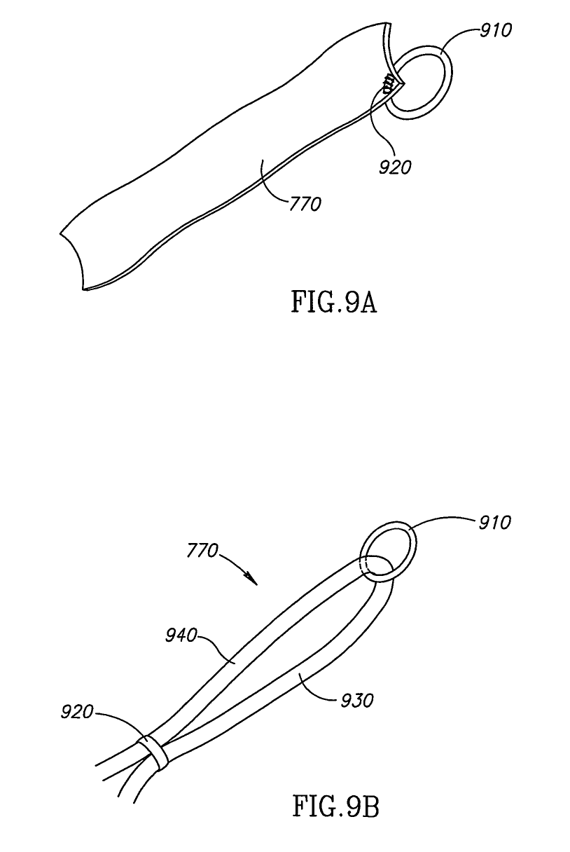

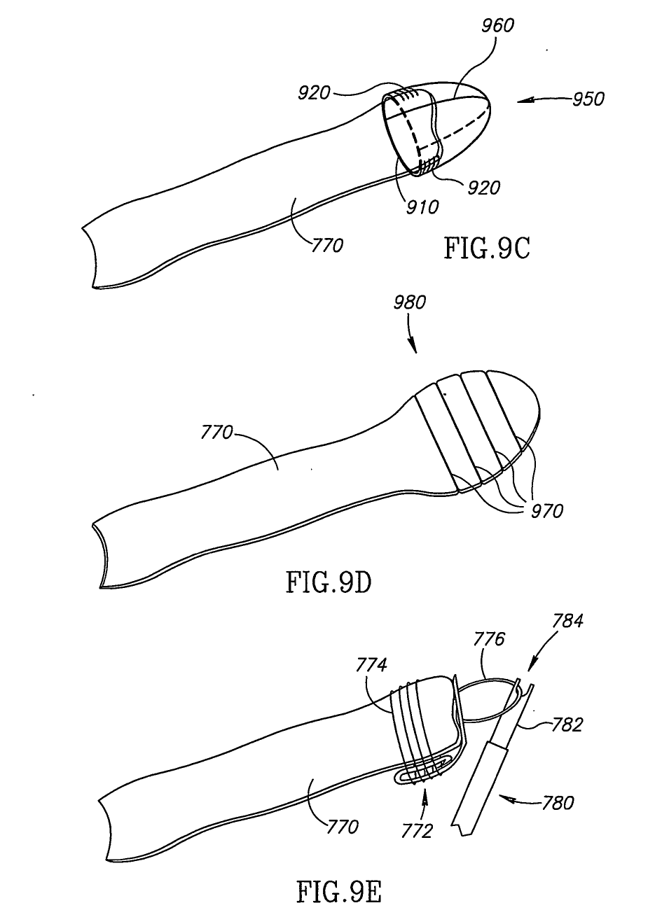

FIGS. 9A to 9E are schematic illustration of grafts with auxiliary anchoring, according to some exemplary embodiments of the invention. In some embodiments of the invention, the auxiliary anchoring device is prepared from a graft with bone attached to the end. Alternatively, the auxiliary anchoring device is prepared from a graft without bone attached, at least at that end.

FIG. 9A shows a ring 910 connected by sutures 920 (other means, such as a gap in the ring, may be used instead) to an end of graft 770 to enhance anchoring of graft 770 in expansion region 160. In some embodiments of the invention, ring 910 is an oval ring so that it can be inserted with the shorter radius perpendicular to the insertion axis. Optionally, ring 910 can be rotated once positioned in expansion region 160 so the longer radius is perpendicular to tunnel 190 to prevent it from sliding out. In some embodiments of the invention, fixation material is added around ring 910 to prevent it from being released. In some embodiments of the invention, ring 910 is connected with glue to the walls of expansion region 160. Alternatively or additionally, ring 910 is elastic, super-elastic or shape-memory so that it can be pushed in hole 190 and then deform (or re-form) to a different shape.

FIG. 9B illustrates graft 770 folded over ring 910 forming two strands 930 and 940. Optionally, the strands 930 and 940 are sutured together with one or more sutures 920 (or adhesive or other means), to keep the strands from sliding off ring 910.

FIG. 9C illustrates an anchor 950 with a wire frame structure, for example a cup-shaped structure. Optionally, anchor 950 comprises crossed arcs 960, which rise out of a plane formed by ring 910. In an exemplary embodiment of the invention, anchor 950 is connected with sutures 920 to graft 770. Optionally, anchor 950 is inserted into expansion region 160 in a folded form and it unfolds when reaching expansion region 160. Once located in expansion region 160, anchor 950 is optionally filled and/or surrounded with a fixation material to prevent it from being able to slide out through tunnel 190.

In some embodiments of the invention, the anchor devices, for example, ring 910 and crossed arcs 960 are comprised of metal. Alternatively, the anchor devices are comprised of polymer, bone, bone substitute, biodegradable materials or other suitable materials known in the art.

FIG. 9D illustrates an anchor 980 created by forming a thickening in graft 770 itself. Optionally, an end of graft 770 is folded over and sutured with sutures 970 to keep it from unfolding. In an exemplary embodiment of the invention, anchor 980 is inserted to expansion region 160 and fixated by adding a filling material as described above.

FIG. 9E shows an alternative where graft 770 is folded over at a section 772 and closed with a suture 774. A suture loop 776 is optionally provided for guiding of graft 770 into expansion region 160. Optionally, suture loop 776 is used to pull graft 770 using hole 161 (FIG. 1) for a pull wire or thread. Alternatively or additionally, as shown, loop 776 is engaged by a notched end 784 of an extension 782 of a graft holding device 780. Other loop holding methods may be used.

In an exemplary embodiment of the invention, the thickening of the end of graft 770 increases it cross-sectional area by at least 20%, 50%, 100%, 150%, 200% or more compared to an un-thickened graft. Optionally, the graft end is made to have a dimension greater than the diameter of hole 170, optionally with a cross-section greater than that of hole 170. Optionally, the thickened graft is 0.5 mm greater in thickness or with dimension than the diameter of hole 170. Optionally, the graft end is 10%, 20%, 40% or more greater in cross-section or diameter than hole 170. Optionally, the end is compressed during insertion.

In an exemplary embodiment of the invention, graft 770 is thickened at an end by mounting it in a plug. Optionally graft 770 is glued to the plug and mounted in a lumen thereon or in a groove thereon. Optionally, the plug is resilient enough to create expansion region 160 when the plug is released. Optionally, the plug is pushed or pulled through hole 170, while in a tube, which prevents its premature expansion and/or friction with the wall of hole 170.

In an exemplary embodiment of the invention, an anchoring aid is formed on the end on graft 770 using a settable material such as bone cement, glue or adhesive. Optionally, the material sets completely before insertion of graft 770. Optionally, the material sets to a condition that is not rigid, so that the aid can be distorted for insertion through hole 170, and then expand in expansion region 160. Alternatively, the anchoring aid is tacky when inserted into region 160 (or hole 170), for example to provide some adhesion to bone. Alternatively, the material sets completely, to a rigid condition, before insertion.

Optionally, a second layer of fixation material is applied to the anchoring aid before insertion and/or injected into hole 170 and/or expansion region 160.

In an exemplary embodiment of the invention, the anchoring aid is formed by applying a layer of cement to graft 770. Optionally, the graft is placed in a mold, so that the final shape of the anchoring aid can be set. In an exemplary embodiment of the invention, a mold comprises a flat hinged element having two leaves that can be brought into opposing condition, with a form defined on opposite leaves. Various types of molds known in the rat for resins, waxes and plastics may be used. Optionally, the mold dissolves in water (e.g., is made of sugar), for removal. Alternatively, the mold is coated or is made of a material t which the cement does not adhere. Optionally, a set of molds of different sizes and/or shapes are provided, for example in kit form, for example for different applications or hole sizes.

In an exemplary embodiment of the invention, the anchoring aid serves to increase friction between the graft and the fixation material. Optionally, the anchoring aid is roughened and/or includes one or more projections and/or indentations, for engaging the fixation material.

In an exemplary embodiment of the invention, the anchoring aid serves to engage expansion region 160 and/or hole 170. In an exemplary embodiment of the invention, the mold defines relatively longer projections (e.g., 1-2 mm). Optionally, additional materials are added to the mold, for example, fibers, stiffening or softening elements, bio-active materials or drug-eluting elements. Such elements may have a diameter, for example, of between 0.5 and 1 mm.

In an exemplary embodiment of the invention, the anchoring aid increases the graft cross-sectional extent by 1, 2, 3, 4 mm or more. Optionally, the increase in cross-sectional area is at least 20%, 40%, 60%, 100% or more. Optionally, the increased cross-section is enough to make the anchoring aid have a dimension greater than the diameter of hole 170 and aid in or prevent retraction.

Optionally, the anchoring aid is molded to include one or more apertures, for example for a K-wire, for a pushing insertion device (such as device 730) or for a pulling wire.

Optionally, the graft is treated at the operating theater. Alternatively, graft material may be treated as described herein at a manufacturing facility or prior to an operation.

In an exemplary embodiment of the invention, cement is applied by dipping sad graft end in cement. Optionally, extraneous material added by dipping or molding is removed manually during or after setting. Optionally, the cement is polished and/or sterilized after application.

General

It will be appreciated that the above described methods may be varied in many ways, including, changing the type of anchor used and/or materials used in the system. It should also be appreciated that the above described description of methods and apparatus are to be interpreted as including apparatus for carrying out the methods and methods of using the apparatus.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. For example, some features are described with regard to one void expansion device and may also be provided in other void expansion devices. Variations of embodiments described will occur to persons of the art.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Also included within the scope of the invention are surgical kits including sterile equipment and including one, two or more of a replacement graft, graft anchor aid, bone cement, plug, graft inserter, drill and/or expansion forming device. Section headings are provided for navigation and should not necessarily be considered as limiting the scope of the invention or description. The scope of the invention is limited only by the elements and limitations as used in the claims. When used in the following claims, the terms “comprise”, “include”, “have” and their conjugates mean “including but not limited to”.

1. A device for drilling a tunnel in a bone and forming expansion regions in the-tunnel having a given lateral dimension, comprising:
an elongate body having an axis;
a head mounted at a distal end of the body:
a sleeve including an inner lumen adapted to accommodate the body with the head protruding from a distal end of the sleeve and adapted to fit through the tunnel; and
characterized by having two operational modes, a tunnel drilling mode in which the sleeve and the head are concentric and a reaming mode in which the sleeve and the head are eccentric;
wherein the drilling mode is suitable for drilling a tunnel in a bone and the reaming mode is suitable for forming expansion regions in a tunnel.

2-6. (canceled)

7. A device according to claim 1, wherein said head is adapted to be rotationally locked to said sleeve.

8. A device according to claim 1, wherein said head has a rotationally limited motion relative to said sleeve.

9. A device according to claim 1, comprising an interlock which selectively rotationally locks said body to said sleeve.

10. A device according to claim 9, wherein said interlock includes a plurality of selectable interlocking settings.

11. A device according to claim 9, wherein said interlock is directionally selective in at least two positions thereof preventing relative rotational motion one only in one rotation direction and one only in an opposite rotation direction.

12. A device according to claim 1, wherein said sleeve has a lumen sized to receive said body and wherein said lumen is off-axis of an axis of said sleeve.

13-17. (canceled)

18. A device according to claim 1, wherein said head is adapted to compact cancellous bone in said tunnel to cause said expansion regions.

19-23. (canceled)

24. A device according to claim 1, wherein said head is adapted to selectively drill or ream depending on a rotation direction of said head.

25. A device according to claim 1, wherein said head is adapted to selectively drill or ream depending on an eccentricity of rotation of said head.

26-28. (canceled)

29. A device according to claim 1, comprising a plurality of depth indicating axial markings.

30. A device according to claim 1, comprising an axially positionable depth limiter.

31. (canceled)

32. A head adapted for drilling a tunnel and for widening an existing tunnel, comprising:
(a) at least one drill cutting edge arranged for cutting bone in a forward pointing direction; and
(b) at least one bone cutting edge arranged for cutting the bone transverse to the forward pointing direction and arranged in a general axial direction;
wherein the head can be set in a first configuration and a second configuration;
wherein the first configuration positions the head so that the bone cutting edge does not cut transverse to the forward pointing direction; and
wherein the second configuration positions the head so that the bone cutting edge cuts transverse to the forward pointing direction.

33. A head according to claim 32, comprising at least a second substantially axially arranged bone cutting edge for adapted for cutting at an opposite rotation direction of said head than a rotation direction for which said head drills said tunnel.

34. A head according to claim 32, wherein said head is mounted off-center on an elongate rod.

35. A head according to claim 32, wherein said head is cannulated.

36. A method of fixating soft tissue to bone, comprising:
(a) forming a tunnel in said bone;
(b) forming a radial expansion region in an inner segment of said tunnel;
(c) inserting soft tissue into said expansion region via said tunnel; and
(d) employing a fixation material capable of setting to fixate said soft tissue in said expansion region of said tunnel.

37. A method according claim 36, wherein said soft tissue comprises a ligament or a tendon or a ligament or a tendon replacement graft.

38-51. (canceled)

52. A method according to claim 36, wherein a same tool is employed for the forming a tunnel and for the forming of the expansion region.

53-61. (canceled)

62. A method according to claim 37, wherein said ligament is an anterior cruciate ligament (ACL).

63-64. (canceled)

65. A method of pre-treating graft material to facilitate fixating to bone, comprising:
(a) providing a graft material suitable to be used for a ligament or tendon; and
(b) increasing a cross-section of said graft at at least one end thereof by adhering a flowable material capable of setting to a hardened condition to said graft.

66-67. (canceled)

68. A method according to claim 65, wherein increasing a cross-section further comprises folding said graft.

69. (canceled)

70. A method according to claim 65, wherein increasing a cross-section comprises dipping said graft material in a settable material.

71-72. (canceled)

73. A kit for fixation of soft tissue to bone comprising:
(a) a drill adapted for formation of a tunnel in a bone;
(b) a reamer adapted to widen a portion of the tunnel;
(c) an insertion tool adapted to engage a portion of a soft tissue and convey the engaged portion through the tunnel to the widened portion;
(d) a quantity of setting material; and
(e) an applicator adapted to deliver at least a portion of the setting material to the portion of the soft tissue in the widened portion of the tunnel.

74. A kit according to claim 73, wherein the drill and the reamer are supplied as a single device.

75. A kit according to claim 73, wherein the insertion tool participates in the delivery of the at least a portion of the setting material.

76. A kit according to claim 73, wherein the setting material is biodegradable.

77. A graft insertion tool, the tool comprising:
(a) a body characterized by a proximal end and a distal end, the distal end adapted for insertion into a tunnel drilled in a bone;
(b) a distal mechanism adapted to engage a portion of a soft tissue and conveying the engaged portion through the tunnel to a widened portion thereof; and
(c) a delivery mechanism adapted to deliver a setting material to the portion of the soft tissue in the widened portion of the tunnel.

78. A method according to claim 68 wherein said folding includes tying.

79. A device according to claim 1, wherein said head is adapted to form the expansion region, the expansion region characterized by a larger diameter than the diameter of the tunnel.

80. A device according to claim 1, wherein one of the two operational modes is selected automatically.

81. A device according to claim 80, wherein one of the two operational modes is selected by axial motion of the head.

82. A method according claim 36, wherein said fixation material comprises a bone void filler.

83. A method according claim 36, wherein said fixation material comprises a bone cement.

84. A method according claim 36, wherein said fixation material comprises a material which expands when brought in contact with a humid environment.

85. A method according claim 36, wherein said fixation material comprises biodegradable material.
